(19) United States
(12) Patent Application Publication
(10) Pub. No.: US 2009/0090030 A1
(43) Pub. Date: Apr. 9, 2009

(54) Article of manufacture for an orthotic with a heel anchor

(76) Inventors: Daniel Morong, Carlsbad, CA (US); Marilyn Morong, Carlsbad, CA (US)

Correspondence Address: HARRY V. MCGAHEY JR., 1532 SIXTH AVENUE, SAN DIEGO, CA, 92101, US

(21) Appl. No.: 11/973,513

(22) Filed: Oct. 9, 2007

(51) Int. Cl. A43B 7/14; A61F 5/14

(52) U.S. Cl. 36/172; 36/140; 36/145

(57) ABSTRACT

An article of manufacture for an Orthotic arch support utilizing triangular shaped heel anchors to minimize slippage inside the shoe when it is worn.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of types of orthotics which are more commonly known as “arch supports” and more specifically to an article of manufacture for an Orthotic with a Heel Anchor and specially shaped Girders placed on the bottom to strengthen it.

There is a need for orthotics which do not slip inside the shoe where they are placed. The invention herein disclosed meets that need in a very effective way.

Historical attempts to address some or all of the limitations in prior patented orthotics have been numerous. Some examples of relevant art include for utility patents: U.S. Pat. No. 5,896,677, and US application 2006/0000114; U.S. Design Pat. Nos. D399,646, D 511,613, D 424,289, D 509,951, D 273,530, D 407,543 and D 474,881; and U.S. Utility Pat. Nos. U.S. 399,042 and U.S. 485,426,

Orthotic arch supports tend to slip inside the wearer's shoe when worn. They also tend to bend and break under the stress and heat of use by the wearer. The invention herein described contains a plurality of triangular shaped heel anchors to minimize slippage inside the shoe when it is worn. The subject invention also has circular spine girders and border rectangular girders which give it greater strength with more flexibility than other orthotics currently in the art.

### BRIEF SUMMARY OF THE INVENTION

The primary object of the invention is To provide a better orthotic for arch support purposes which does not slip inside the wearer's shoe.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed an article of manufacture for an Orthotic with a Heel Anchor comprising: an orthotic arch support, a plurality of triangular shaped anchor wedges on the bottom heel of the orthotic arch support, and a series of center circular spine girder designs and border rectangular shaped girder designs for strength.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

Note: Each figure is a view of one orthotic for one shoe. The orthotic to be placed in the opposite shoe would be a mirror image of the view shown.

FIG. 1 is a top view of an orthotic with triangular heel designs for use in a shoe showing our new design;

FIG. 2 is a bottom view thereof;

FIG. 3 is a side view thereof with the orthotic upside down;

FIG. 4 is a side view thereof with the orthotic right side up;

FIG. 5 is a front view thereof,

FIG. 6 is a rear view thereof;

FIG. 7 is a bottom perspective view thereof;

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Note: Each figure is a view of one orthotic for one shoe. The orthotic to be placed in the opposite shoe would be a mirror image of the view shown.

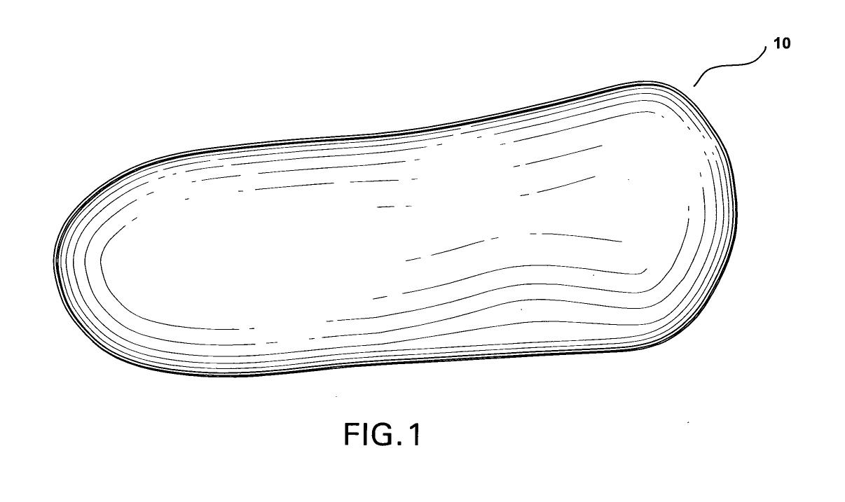

FIG. 1 is a top view of an orthotic (10) with triangular heel designs for use in a shoe showing our new design. None of the triangular heel designs can be seen from this top view.

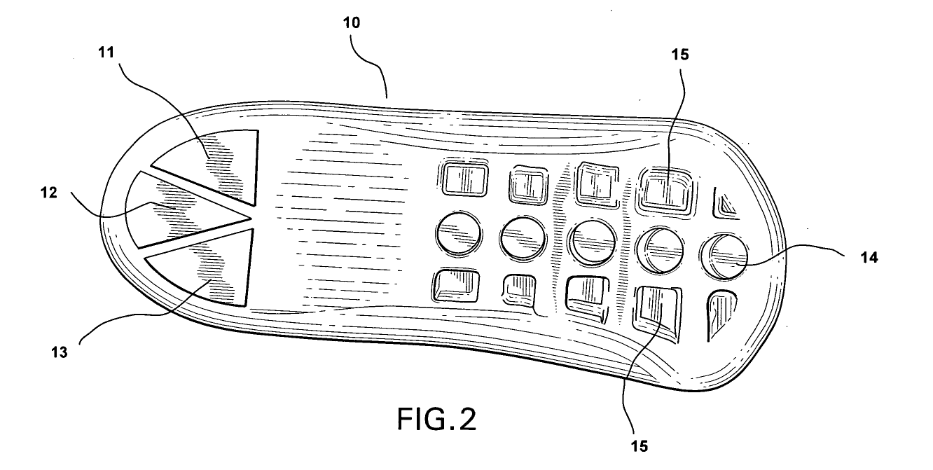

FIG. 2 is a bottom view of the orthotic (10) showing the preferred embodiment. The preferred embodiment is comprised of three triangular shaped Anchor Wedge Shapes (11), (12) and (13) extending approximately 1/16″ from the bottom of the orthotic. The Middle Triangular Anchor Wedge (12) has its acute angle facing towards the front of the orthotic, while the Exterior Triangular Anchor Wedge (11) on the outside of the heel and the Interior Triangular Anchor Wedge (13) on the inside of the heel have their acute angle facing towards the rear of the orthotic. In describing the orthotic, the “rear” area is deemed to be the area where the heel is placed, while the “front” area is the area towards where the toes are located when the orthotic is worn. This figure also shows the Center Circular Spine Girder Design (14) and the Border Rectangular Girders (15) which are located in each side of the Center Circular Spine Girders (14). There is more polymer material comprising the orthotic surrounding the round orifice designs (14) than the rectangular orifice designs (15). This extra polymer material down the center section of the orthotic provides additional strength and durability. This is an area which if not properly reinforced will flatten out—thus eliminating the objective of wearing the orthotic. This extra polymer surrounding the round holes and where they are placed (down the middle) allows a 6 degree rise in the arc for a stable flexing motion. The depth of round orifices (14) and the square orifices (15) in the grid pattern design towards the front of the orthotic are based on the rise of the arch for a particular orthotic. The “arc” of an orthotic is the rise in the arch portion of the orthotic from a base level. As the arc rises, more material is able to be used to maintain the integrity of the arc, while still maintaining the desired amount of flexibility. The depth in the orifices in the grid allows for the required flexing motion, as the individual steps down on the orthotic. The depth of the orifices vary, from the first orifice at the top of the orthotic to the last vertical orifice in the orthotic, this variance is adjusted to maintain the desired rise in the arc necessary to fit the wearer and adjust his or her arch height accordingly.

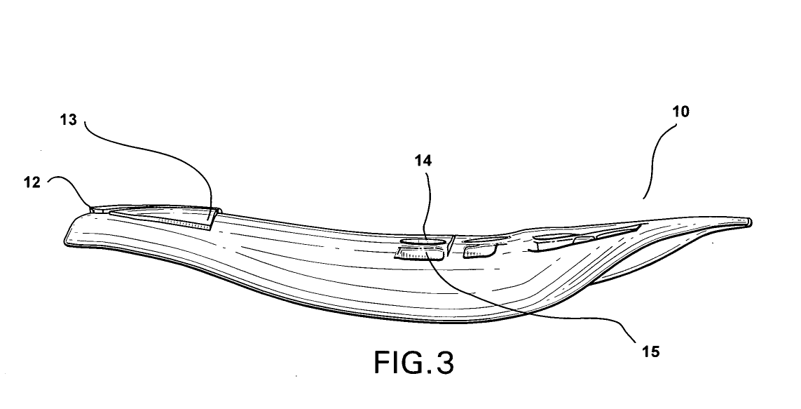

FIG. 3 is a side view thereof with the orthotic (10) upside down. Part of the Middle Triangular Anchor Wedge (12) can be seen protruding up from the base of the upside down orthotic. The wide base of the Interior Triangular Anchor Wedge (13) can also be seen on the left of the figure, as can the Border Rectangular Girder design (15) for one side of the orthotic (10).

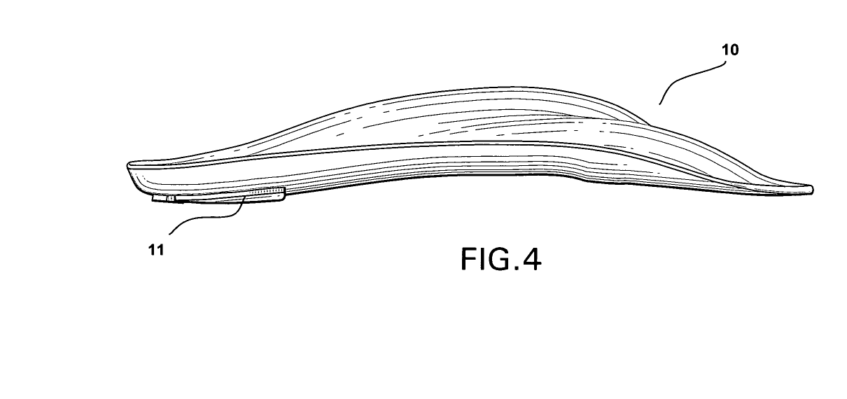

FIG. 4 is a side view thereof with the orthotic right side up. In this view the Exterior Triangular Anchor Wedge (11) can be seen protruding down from the base of the orthotic (10). This Exterior Triangular Anchor Wedge (11), by protruding downwards tends to engage in the soft sole of the shoe the orthotic is worn in, thereby preventing and minimizing slippage when it is worn by the user.

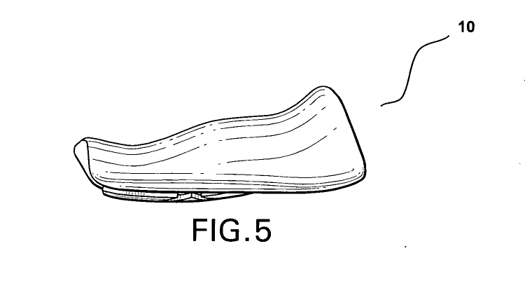

FIG. 5 is a front view of the orthotic. Part of the Anchor Wedges can be seen protruding from the lower base of orthotic.

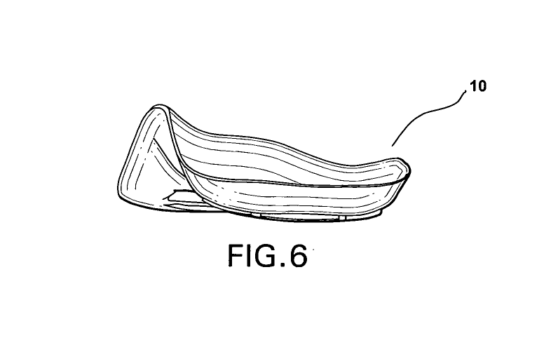

FIG. 6 is a rear view of the orthotic. One of the Border Rectangular Girder designs towards the front can be seen.

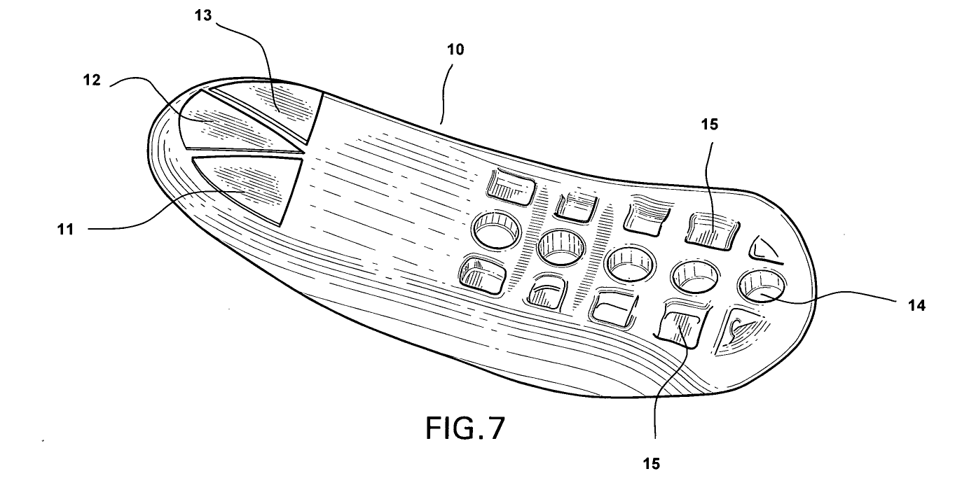

FIG. 7 is a bottom perspective view thereof. As in FIG. 2, there are shown three triangular shaped Anchor Wedge Shapes (11), (12) and (13) extending 1/16″ from the bottom of the orthotic. The Middle Triangular Anchor Wedge (12) can been with its acute angle facing towards the front of the orthotic, while the Exterior Triangular Anchor Wedge (11) and the Interior Triangular Anchor Wedge (13) can be seen with their acute angle facing towards the rear of the orthotic. Like FIG. 2, this figure also shows the Center Circular Spine Girder designs (14) and the Border Rectangular Girder designs (15) which are located in each side of the Center Circular Spine Girder designs (14).

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. An article of manufacture for an orthotic with a heel anchor comprising:
an orthotic arch support; and
a plurality of triangular shaped anchor wedges on the bottom heel of the orthotic arch support.

2. An article of manufacture for an orthotic with a heel anchor as claimed in claim 1 wherein said triangular shaped anchor wedges are comprised of:
an exterior triangular anchor wedge on the bottom heel of the orthotic arch support placed towards the outside of the wearer's heel;
a middle triangular anchor wedge adjacent to the exterior triangular anchor wedge; and
an interior triangular anchor wedge adjacent to the middle triangular anchor wedge.

3. An article of manufacture for an Orthotic with a Heel Anchor as claimed in claim 1 wherein said arch support contains center circular spine girders and border rectangular girders on the lower bottom of the orthotic.

4. An orthotic with a plurality of triangular shaped wedges protruding from the bottom of the heel area.

5. The orthotic in claim 4 wherein a plurality of circular orifices are located on the longitudinal axis of the orthotic.

6. The orthotic in claim 4 wherein there are a plurality of rectangular impressions located along each side of the longitudinal axis of the circular impressions.

7. The orthotic in claim 4 wherein the triangular shaped wedges protrude between 1/32″ and ⅛″ from the bottom of the orthotic.

8. The orthotic in claim 4 wherein there are three triangular shaped wedges protruding from the bottom of the heel area.

9. The orthotic in claim 4 wherein the triangular shaped wedge in along the center most longitudinal axis has the acute angle facing towards the front of the orthotic.

10. The orthotic in claim 4 wherein there is a triangular shaped wedge located along each side of the longitudinal axis of the triangular shaped wedge along the center most longitudinal axis.

11. The orthotic in claim 10 wherein the triangular shaped wedges located along each side of the longitudinal axis of the triangular shaped wedge along the center most longitudinal axis have their acute angles facing towards the rear of the orthotic
